SEQUENCE LISTING (6 sequences)

SEQ ID NO: 1 - DNA, 845 bp, Corynebacterium species
  misc_feature: “n” positions designate restriction endonuclease recognition site
nnnatgacagttcccagcatcgtgctcaacgacggcaattccattccccagctcgggtacggcgtcttcaaggtgccgccggcggacacccagcgcgccgtcgaggaagcgctcgaagtcggctaccggcacatcgacaccgcggcgatctacggaaacgaagaaggcgtcggcgccgcgatcgcggcgagcggcatcgcgcgcgacgacctgttcatcacgacgaagctctggaacgatcgccacgacggcgatgagcccgctgcagcgatcgccgagagcctcgcgaagctggcactcgatcaggtcgacctgtacctcgtgcactggccgacgcccgccgccgacaactacgtgcacgcgtgggagaagatgatcgagcttcgcgcagccggtctcacccgcagcatcggcgtctcgaaccacctcgtgccgcacctcgagcgcatcgtcgccgccaccggcgtcgtgccggcggtgaaccagatcgagctgcaccccgcctaccagcagcgcgagatcaccgactgggccgccgcccacgacgtgaagatcgaatcgtgggggccgctcggtcagggcaagtacgacctcttcggcgccgagcccgtcactgcggctgccgccgcccacggcaagaccccggcgcaggccgtgctccgttggcacctgcagaagggtttcgtggtcttcccgaagtcggtccgccgcgagcgcctcgaagagaacctcgacgtgttcgacttcgacctcaccgacaccgagatcgccgcgatcgacgcgatggatccgggcgacggttcgggtcgcgtgagcgcacaccccgatgaggtcgactgannnnn

SEQ ID NO: 2 - DNA, 845 bp, Corynebacterium species
catatgaccgttccgtctatcgttctgaacgacggtaactctatcccgcagctgggttacggtgttttcaaagttccgccggctgacacccagcgtgctgttgaagaagctctggaagttggttaccgtcacatcgacaccgctgctatctacggcaacgaagaaggtgttggtgctgctatcgctgcttctggtatcgctcgtgacgacctgttcatcaccaccaaactgtggaacgaccgccacgacggtgacgaaccggctgctgctatcgctgaatctctggctaaactggctctggatcaggttgacctgtacctggttcactggccgaccccggctgctgacaactacgttcacgcttgggaaaaaatgatcgaactgcgtgctgctggtctgacccgttctatcggtgtttctaaccacctggttccgcacctggaacgtatcgttgctgctaccggtgttgttccggctgttaaccagatcgaactgcacccggcttaccagcagcgtgaaatcaccgactgggctgctgctcacgacgttaaaatcgaatcttggggtccgctgggtcagggtaaatacgacctgttcggtgctgaaccggtaaccgctgctgctgctgctcacggtaaaaccccggctcaggctgttctgcgttggcacctgcagaaaggtttcgttgttttcccgaaatctgttcgtcgtgaacgtctggaagaaaacctggacgttttcgacttcgacctgaccgacaccgaaatcgctgctatcgacgctatggatccgggcgacggttctggtcgtgtttctgctcacccggacgaagttgactgaagctt

SEQ ID NO: 3 - DNA, 843 bp, Corynebacterium species
  misc_feature: “n” positions at both ends of sequence represent restriction endonuclease recognition sites; “n” positions at residues 49-51, and 55-57 represent areas of disagreement in the published sequence f or wild type DKGR-B between Sonoyama and Powers, however, both published sequences encode the same amino acid
nnnatgccgaacatccccaccatcagcctcaacgacggacgccccttcnnngagnnngggctcggcacgtacaacctgcgcggcgacgagggggtcgcggccatggtcgccgcgatcgactcgggctaccgcctgctcgacacggcggtgaactacgagaacgagagcgaggtcggccgagcggtgcgcgcgagcagcgtcgatcgcgacgagctcatcgtggcgagcaagatcccgggccgccagcacgggcgcgccgaggcggtcgacagcatccgcggatcgctcgaccggctggggctcgacgtgatcgacctgcagctgatccactggccgaaccccagcgtgggccggtggctcgacacctggcgcggcatgatcgacgcgcgcgaggcgggcctggtccgctcgatcggcgtctcgaacttcaccgagccgatgctgaagaccctcatcgacgagaccggggtcacacccgcggtcaaccaggtcgagctccacccgtacttcccccaggcggcgctgcgcgcgttccacgacgagcacggcatccgcaccgagagctggagcccgctcgcccggcgcagcgagctgctcaccgagcagctgctgcaggagctggcggtcgtctacggagtgacgccgacgcaggtggtgctgcggtggcacgtgcagctcggcagcaccccgatccccaagtccgccgaccccgatcgccagcgcgagaacgccgatgtgttcggcttcgccctcaccgccgaccaggtcgatgcgatctcgggcctcgagcgcgggcggctctgggacggcgaccccgacacgcacgaagagatgtagnnnnnn

SEQ ID NO: 4 - DNA, 843 bp, Corynebacterium species
catatgccgaacatcccgaccatctctctgaacgacggtcgtccgttcccggaactgggtctgggtacctacaacctgcgtggtgacgaaggtgttgctgctatggttgctgctatcgactctggttaccgtctgctggacaccgctgttaactacgaaaacgaatctgaagttggtcgtgctgttcgtgcttcttctgttgaccgtgacgaactgatcgttgcttctaaaatcccgggtcgtcagcacggtcgtgctgaagctgttgactctatccgtggttctctggaccgtctgggtctggacgttatcgacctgcagctgatccactggccgaacccgtctgttggtcgttggctggacacctggcgtggtatgatcgacgctcgtgaagctggtctggttcgttctatcggtgtctctaacttcaccgaaccgatgctgaaaaccctgatcgacgaaaccggtgttaccccggctgttaaccaggttgaactgcacccgtacttcccgcaggctgctctgcgtgctttccacgacgaacacggtatccgtaccgaatcttggtctccgctggctcgtcgttctgaactgctgaccgaacagctgctgcaggaactggctgttgtttacggtgttaccccgacccaggttgttctgcgttggcacgttcagctgggttctaccccgatcccgaaatctgctgacccggaccgtcagcgtgaaaacgcagacgttttcggtttcgctctgaccgctgaccaggttgacgctatctctggtctggaacgtggtcgtctgtgggacggtgacccggacacccacgaagaaatgtagaagctt

SEQ ID NO: 5 - protein, 277 aa, Corynebacterium species
MTVPSIVLNDGNSIPQLGYGVFKVPPADTQRAVEEALEVGYRHIDTAAIYGNEEGVGAAIAASGIARDDLFITTKLWNDRHDGDEPAAAIAESLAKLALDQVDLYLVHWPTPAADNYVHAWEKMIELRAAGLTRSIGVSNHLVPHLERIVAATGVVPAVNQELHPAYQQREITDWAAAHDVKIESWGPLGQGKYDLFGAEPVTAAAAAHGKTPAQAVLRWHLQKGFVVFPKSVRRERLEENLDVFDFDLTDTEIAAIDAMDPGDGSGRVSAHPDEVD

SEQ ID NO: 6 - protein, 277 aa, Corynebacterium species
MPNIPTISLNDGRPFPELGLGTYNLRGDEGVAAMVAAIDSGYRLLDTAVNYENESEVGRAVRASSVDRDELIVASKIPGRQHGRAEAVDSIRGSLDRLGLDVIDLQLIHWPNPSVGRWLDTWRGMIDAREAGLVRSIGVSNFTEPMLKTLIDETGVTPAVNQVELHPYFPQAALRAFHDEHGIRTESWSPLARRSELLTEQLLQELAVVYGVTPTQVVLRWHVQLGSTPIPKSADPDRQRENADVFGFALTADQVDAISGLERGRLWDGDPDTHEEM

(19) United States
(12) Patent Application Publication
(10) Pub. No.: US 2002/0146731 A1
(43) Pub. Date: Oct. 10, 2002

(54) Synthetic nucleic acid sequences for 2,5-diketo-D-gluconic acid reductases and associated methods

(76) Inventors: Gulsah Sanli, Tallahassee, FL (US); Sachiko I. Blaber, Tallahassee, FL (US); Michael Blaber, Tallahassee, FL (US)

Correspondence Address: ENRIQUE G. ESTEVEZ, Allen, Dyer, Doppelt, Milbrath & Gilchrist, P.A., P O Box 3791, Orlando, FL, 32802-3791, US

(21) Appl. No.: 10/037,633

(22) Filed: Jan. 3, 2002

Related U.S. Application Data
(60) Provisional application No. 60/259,527, filed on Jan. 3, 2001.

(51) Int. Cl. C12Q 1/68; C07H 21/04; C12P 19/34; C12N 9/22; C12P 21/02; C12N 5/06

(52) U.S. Cl. 435/6; 435/91.2; 435/199; 435/69.1; 435/325; 435/320.1; 536/23.2

(57) ABSTRACT

An isolated nucleic acid comprises a degenerate variant of the nucleotide sequence of wild-type DKGR A having a GC content from about 55% to about 67%, and an isolated nucleic acid comprises a degenerate variant of the nucleotide sequence of wild-type DKGR B having a GC content from about 56% to about 70%. A method of making a polypeptide, comprises culturing an isolated cell having a nucleic acid degenerate variant of the nucleotide sequence of SEQ ID NO:1 having a GC content of from about 55% to about 67%, or of the nucleotide sequence of SEQ ID NO:3 having a GC content of from about 56% to about 70%, and an expression vector therefor operably linked to an expression control sequence, wherein culturing is effected under conditions permitting expression of said nucleic acid so as to produce a polypeptide encoded thereby.

### RELATED APPLICATION

This application claims priority from co-pending provisional application Serial No. 60/259,527 which was filed on Jan. 3, 2001, and which is incorporated herein by reference in its entirety.

### FIELD OF THE INVENTION

The present invention relates to the field of synthetic genes and, more particularly, to a synthetic or isolated nucleic acid sequence encoding 2,5-diketo-D-gluconic acid reductases (DKGR A and DKGR B), which are Corynebacterium polypeptides having a wild-type amino acid sequence, yet demonstrating enhanced heterologous expression and enhanced efficiency in polymerase-based methodologies, properties not possessed by the natural wild-type gene.

### BACKGROUND OF THE INVENTION

Corynebacterium species codon usage exhibits an overall GC content of 67%, and a wobble-position GC content of 88%. Escherichia coli, on the other hand has an overall GC content of 51%, and a wobble-position GC content of 55%. The high GC content of wild type Corynebacterium nucleic acids results in an unfavorable codon preference for heterologous expression, particularly in enteric bacteria, and in Escherichia coli especially, and can also present difficulties for polymerase-based manipulations due to secondary-structure effects.

Since these characteristics are due primarily to base pairings at the wobble-position of a tRNA anticodon, synthetic genes might be designed to reduce these problems and yet retain the wild-type amino acid sequence. If feasible, such genes could eliminate the need for special additives or bases during in vitro polymerase-based manipulation and for mutant host strains containing uncommon tRNA's for improved heterologous expression.

The enzymes 2,5-diketo-D-gluconic acid reductases (2,5-DKGR; E.C. 1.1.1.-) from Corynebacterium catalyze the NADPH-dependent reduction of 2,5-diketo-D-gluconic acid (2,5-DKG) to 2-keto-L-gulonic acid (2-KLG) (Sonoyama, Tani et al. 1982). 2-KLG is a key intermediate in the commercial synthesis of L-ascorbic acid (vitamin C) (Anderson, Marks et al. 1985; Miller, Estell et al. 1987; Grindley, Payton et al. 1988). Two variants of this enzyme, 2,5-DKGR A and 2,5-DKGR B, have been identified with 41% identity at the DNA level and 38% identity at the amino acid level (Sonoyama and Kobayashi 1987). Both Corynebacterium genes have high GC content; form A having 68% (Anderson, Marks et al. 1985) and form B having 71% (Grindley, Payton et al. 1988). Sequencing and PCR amplification of the 2,5-DKGR genes have proven problematic (Anderson, Marks et al. 1985; Powers 1996), presumably due to regions of high melting temperature or residual secondary structure in G/C-rich regions of the DNA duplex. Heterologous expression of Corynebacterium 2,5-DKGR A has been demonstrated in Etwinia herbicola (Anderson, Marks et al. 1985), while expression attempts in E.coli have proven unsuccessful (Powers 1996). Heterologous expression of 2,5-DKGR B in E. coli has been reported, but the level of expression was not evaluated (Grindley, Payton et al. 1988).

Analysis of codon statistics for Corynebacterium is limited by a relatively small sample population but indicates that there is an overall bias for G/C residues of 67%, with 67% G/C content in the first position, 45% in the second, and 88% in the wobble-position (Genbank). E coli, on the other hand, has an overall bias for G/C residues of 51%, with 59% G/C content in the first position, 41% in the second, and 55% in the wobble-position (Genbank). Therefore, we proposed that reduction of the G/C content of Corynebacterium genes may be achievable by appropriate substitutions at the wobble-position base, while retaining the corresponding amino acid sequence. We also theorized that such altered genes may exhibit improved properties with regard to polymerase-based manipulations. Furthermore, appropriate alterations at the wobble positions may additionally increase the preferred codon usage for heterologous expression in enteric bacteria.

### SUMMARY OF THE INVENTION

With the foregoing in mind, the present invention advantageously provides a synthetic or isolated nucleic acid comprising a degenerate variant of the nucleic acid sequence of wild-type DKGR A having a GC content from about 55% to about 67%. Additionally, the invention includes an isolated nucleic acid comprising a degenerate variant of the nucleotide sequence of wild-type DKGR B having a GC content from about 56% to about 70%. The invention also includes various methods for making these enzymes, as well as a method of making vitamin C wherein enzymes expressed from these synthetic or isolated nucleic acids are used.

Such synthetic or isolated nucleic acids encoding 2,5-DKGR A and B were designed and assembled in a two-step PCR method (Dillon and Rosen 1990) and their PCR and heterologous expression properties evaluated. Moreover, we evaluated synthetic nucleic acid sequences having reduced wobble-position G/C content using two variants of the enzyme 2,5-diketo-D-gluconic acid reductase (2,5-DKGR A and B) from Corynebacterium. The wild-type genes are refractory to polymerase-based manipulations and exhibit poor heterologous expression in enteric bacteria. The invention herein discloses that a subset of codons for five amino acids (alanine, arginine, glutamate, glycine and valine) provide the greatest contribution to reduction in G/C content at the wobble-position. Furthermore, changes in codons for two amino acids (leucine and proline) enhance bias for expression in enteric bacteria without affecting the overall G/C content. The synthetic nucleic acid sequences disclosed herein are readily amplified using polymerase-based methodologies, and exhibit high levels of heterologous expression in E. coli.

### BRIEF DESCRIPTION OF THE DRAWINGS

Some of the features, advantages, and benefits of the present invention having been stated, others will become apparent as the description proceeds when taken in conjunction with the accompanying drawings in which:

FIG. 1 illustrates the analysis of synthetic 2,5-DKGR A and B nucleic acid sequences by 1% agarose gel electrophoresis, wherein lanes 1, 2, 7 and 8, are DNA size markers; lanes 3 and 5, products of the first PCR step in construction of the synthetic sequences for 2,5-DKGR A and B, respectively; lanes 4 and 6, the end products of the second PCR for synthetic sequences of 2,5-DKGR A and B, respectively; lane 9, DNA size marker; lanes 10 and 11, PCR of wild-type 2,5-DKGR A and B sequences, respectively, using outer primers as described for the second PCR reaction for the synthetic sequences;

FIG. 2 shows the expression of synthetic 2,5-DKGR A and B nucleic acid sequences in pET21 expression vector and E. coli BL21 (IDE3) host, wherein lanes 1 and 6, are molecular weight markers; lanes 2 and 4, are synthetic 2,5-DKGR A and B sequences in pET21 expression vector, respectively, non-induced; lanes 3 and 5, show synthetic sequences for 2,5-DKGR A and B in pET21 expression vector induced by 1 mM isopropyl-b-D-thiogalactopyranoside (IPTG), respectively;

FIG. 3 is a flow diagram illustrating synthesis of vitamin C according to the traditional Reichstein-Grussner process;

FIG. 4 illustrates synthesis of vitamin C according to the tandem fermentation method of Sonoyama; and

FIG. 5 is a diagram of vitamin C synthesis according to the “single bug” method of Anderson.

### DETAILED DESCRIPTION OF THE PREFERRED EMBODIMENT

The present invention will now be described more fully hereinafter with reference to the accompanying drawings, in which preferred embodiments of the invention are shown. This invention may, however, be embodied in many different forms and should not be construed as limited to the illustrated embodiments set forth herein. Rather, these illustrated embodiments are provided so that this disclosure will be thorough and complete, and will fully convey the scope of the invention to those skilled in the art.

Definitions

“Amino acid” refers to all naturally occurring L-.alpha.-amino acids. This definition is meant to include norleucine, ornithine, and homocysteine. The amino acids are identified by their standard single-letter or three-letter designations, as known in the art and shown below:

| | | |
|---|---|---|
| A | Ala | Alanine |
| C | Cys | Cysteine; |
| D | Asp | Aspartic acid; |
| E | Glu | Glutamic acid; |
| F | Phe | Phenylalanine; |
| G | Gly | Glycine; |
| H | His | Histidine; |
| I | Ile | Isoleucine; |
| K | Lys | Lysine; |
| L | Leu | Leucine; |
| M | Met | Methionine; |
| N | Asn | Asparagine; |
| P | Pro | Proline; |
| Q | Gln | Glutamine; |
| R | Arg | Arginine; |
| S | Ser | Serine; |
| T | Thr | Threonine; |
| V | Val | Valine |
| W | Trp | Tryptophan; and |
| Y | Tyr | Tyrosine. |

“Anticodon” means the three-base sequence in tRNA complementary to a codon on mRNA. A nucleotide triplet in a tRNA molecule that aligns with a particular codon in mRNA under the influence of the ribosome, so that the amino acid carried by the tRNA is added to a growing protein chain.

“Codon” is a section of DNA (three nucleotide pairs in length) or RNA (three nucleotides in length) that codes for a single amino acid. A sequence of three RNA or DNA nucleotides that specifies (codes for) either an amino acid or the termination of translation.

“Codon bias” or “codon preference” is the concept that for amino acids which are encoded by several codons, only one or a few are preferred and are used disproportionately in a given host system. They would correspond with tRNAs that are abundant.

“Expression vector” and “vector” are capable of expressing nucleic acid sequences contained therein where such sequences are operably linked to other sequences capable of effecting their expression. It is implied, although not explicitly stated, that expression vectors must be replicable in the host organisms either as episomes or as an integral part of chromosomal nucleic acid. Clearly, a lack of replication would render them effectively inoperable. Accordingly, “vector” or “expression vector” are also given a functional definition. Generally, useful expression vectors also include “plasmids”, which are circular single or double-stranded DNA containing an origin of replication derived from a bacteriophage. These plasmids are not linked to the chromosomes but replicate independently. Other effective vectors commonly used are phage and non-circular DNA. In the present specification, “vector”, “expression vector”, and “plasmid” may be used interchangeably. However, the invention is intended to include such other forms of expression vectors which serve equivalent functions and which are known or which subsequently become known.

“Host”, “host cell”, “cells”, “cell cultures”, “recombinant host cells” and the like may be used interchangeably to designate individual cells, cell lines, cell cultures, and harvested cells which have been or are intended to be transformed with the recombinant vectors of the invention. These terms also include the progeny of the cells originally receiving the vector.

“PCR” or “polymerase-based methodology” are intended to include methods for amplifying specific DNA segments which exploit certain features of DNA replication. For instance replication requires a primer, and specificity is determined by the sequence and size of the primer. The method amplifies specific DNA segments by cycles of template denaturation; primer addition; primer annealing and replication using thermostable DNA polymerase. The degree of amplification achieved is set at a theoretical maximum of 2ˆ N, where N is the number of cycles, eg 20 cycles gives a theoretical 1048576 fold amplification.

“Synthetic” in relation to nucleic acid sequences for the “wild-type” 2,5-DKGR A and DKGR B, refers to a nucleic acid sequence encoding the wild-type amino acid sequence so that enzymatic activity has substantially the same spectrum as the wild-type enzyme, converting 2,5-DKG to 2-KLG. The synthetic nucleic acid sequences, however, contain one or more base substitutions selected in view of the degeneracy of the code to reduce GC content in the sequence, yet to maintain the wild-type amino acid sequence of the polypeptide molecule. The synthetic nucleic acid sequences also demonstrate enhanced efficiency in polymerase-based methodologies, and enhanced heterologous expression in Escherichia coli.

“Transformed” means any process for altering the nucleic acid content of the host. This includes in vitro transformation procedures such as calcium phosphate or DEAE-dextran-mediated transfection, electroporation, nuclear injection, phage infection, or such other means for effecting controlled nucleic acid uptake, as are known in the art.

“tRNA”, also “transfer RNA”, are small RNA molecules that carry amino acids to the ribosome for polymerization into a polypeptide. During translation an amino acid is inserted into a growing polypeptide chain when the anticodon of the tRNA pairs with a complementary codon on the mRNA being translated.

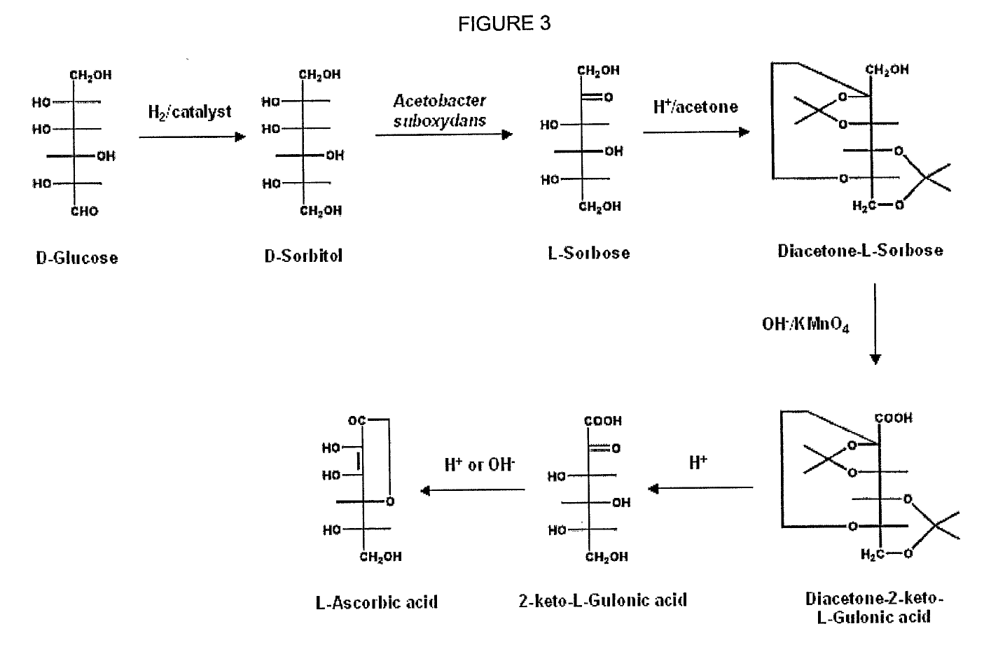

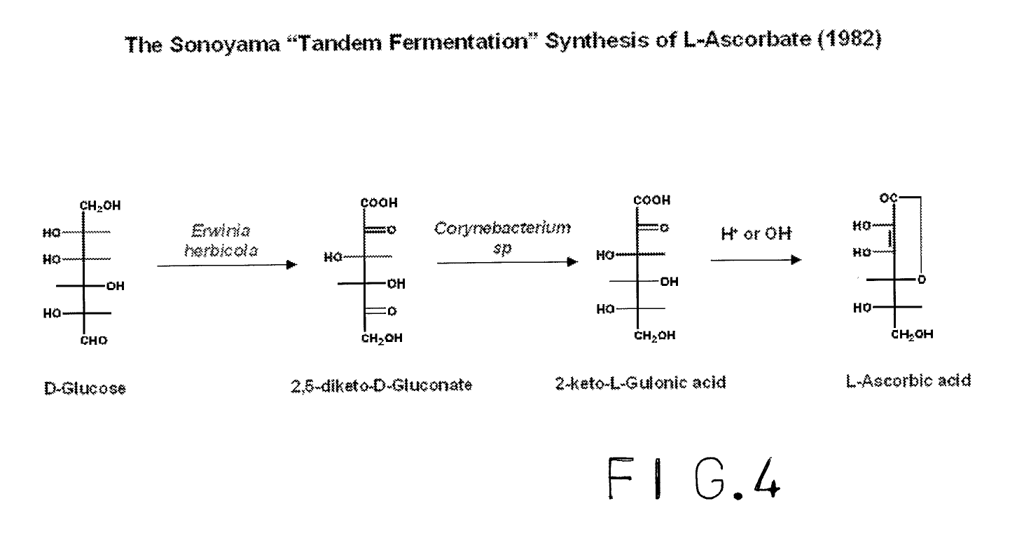

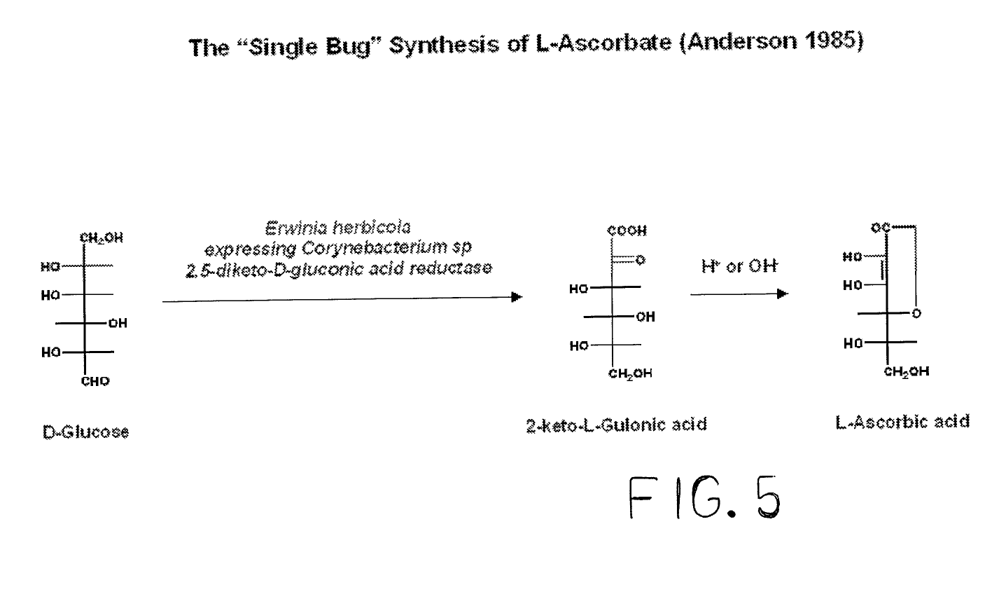

“Vitamin C”, “L-ascorbic acid”, or “ascorbic acid” are used interchangeably herein for the well known and commercially important nutritional supplement generally synthesized according to one of several prior art methods: the traditional Reichstein-Grussner process shown in FIG. 3, wherein 2,5-diketo-D-gluconic acid (2,5-DKG) is not an intermediate; the tandem fermentation method of Sonoyama illustrated in FIG. 4; and the “single bug” method of Anderson shown schematically in FIG. 5, these last two methods both including 2,5-DKG as an intermediate product.

“Wild-type” 2,5-DKGR A or DKGR B refers to a polypeptide, more specifically, an enzyme capable of catalyzing conversion of 2,5-DKG to 2-KLG, a conversion which is stereoselective. The wild-type enzyme is the natural enzyme, before modifications as disclosed herein. The enzyme is obtained from a Corynebacterium species derived from ATCC strain No. 31090 as described in U.S. Pat. No. 5,008,193, which is incorporated herein by reference in its entirety, the amino acid and nucleic acid sequence encoding the wild-type enzyme being described therein.

“Wobble” refers to the ability of certain bases at the third position of an anticodon of tRNA to form hydrogen bonds in various ways, causing alignment with several possible codons. Referring to the reduced constraint of the third base of an anticodon as compared with the other bases thus allowing additional complementary base pairings.

“Wobble position” refers not only to the third base position of an anticodon of tRNA, as described above, but also to a complementary base position along a nucleic acid sequence, for example, DNA and mRNA.

General Methods

A total of 155 codons out of 278 total in 2,5-DKGR A, and 163 codons out of 277 total in 2,5-DKGR B were changed in the design of the synthetic nucleic acid sequences. In 2,5-DKGR A, 116 codon changes result in a decrease in the G/C content, 31 result in no change, and 8 result in an increase in G/C content, as shown in Table 1. In 2,5-DKGR B, 125 codon changes result in a decrease in G/C content, 30 result in no change and 8 result in an increase in G/C content, as shown in Table 2. A total of 154 codon changes out of 155 in 2,5-DGKR A, and a total of 160 codon changes out of 163 in 2,5-DKGR B, result in an increase in the preferred codon bias for the E. coli host. The resulting nucleotide sequences for 2,5-DKGR A and B reduce the overall GC content from 68% to 55% and from 71% to 56%, respectively, and increase the average codon bias for enteric bacteria from 44% to 66% and from 41% to 68% respectively.

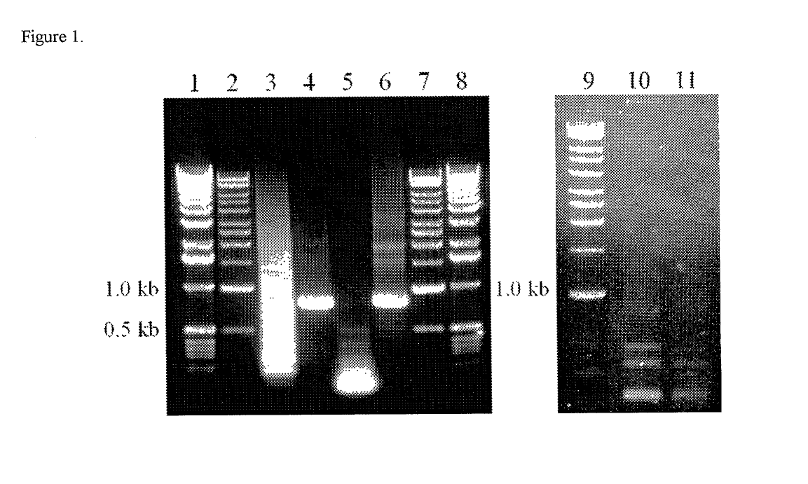

The results of the initial PCR for the construction of the nascent template indicate the presence of several PCR products, most of which are smaller than the desired full-length 2,5-DKGR sequences, as shown in FIG. 1. Nonetheless, the second PCR step, using outer primers, resulted in the production of a DNA product with a size appropriate for the full-length sequences, also seen in FIG. 1. Thus, the initial PCR step resulted in the successful assembly of full-length sequences, in addition to various partial gene fragments. Sequence analysis of the pFASTBAC1 subcloned PCR product indicated two point mutations within the 2,5-DKGR A sequence and one point mutation within the 2,5-DKGR B sequence. Repeated PCR experiments resulted in similar numbers of point mutations, albeit at different locations. The correct synthetic nucleic acid sequences were thus produced by subsequent site-directed mutagenesis upon sequences within the pFASTBAC1 vector. Re-sequencing in the pET-21(+) expression vector confirmed the correct desired sequences.

TABLE 1
The most significant codon substitutions in the
construction of the synthetic 2,5-DKGR A gene.
The relative effects upon codon wobble position
G/C content and bias in relationship to enteric
bacteria codon preference are listed.

| Residue | From | To | ΔWobble G/C | ΔBias |
|---|---|---|---|---|
| ALA | GCG(17), GCC(17) | GCT | −34 | 5.78 |
| ARG | CGC(10), CGG(1) | CGT | −11 | 5.64 |
| GLU | GAG(13) | GAA | −13 | 7.28 |
| GLY | GGC(12), GGG(2) | GGT | −14 | 3.66 |
| LEU | CTC(17) | CTG | 0 | 12.92 |
| LYS | AAG(9) | AAA | −9 | 4.32 |
| PRO | CCC(7) | CCG | 0 | 5.39 |
| SER | AGC(5), TCG(4) | TCT | −9 | 2.8 |
| THR | ACG(3) | ACC | 0 | 1.44 |
| VAL | GTC(11), GTG(12) | GTT | −23 | 9.04 |

TABLE 2
The most significant codon substitutions in the
construction of the synthetic 2,5-DKGR B gene.
The relative effects upon codon wobble position
G/C content and bias in relationship to enteric
bacteria codon preference are listed.

| Residue | From | To | ΔWobble G/C | ΔBias |
|---|---|---|---|---|
| ALA | GCG(14), GCC(7) | GCT | −21 | 3.01 |
| ARG | CGC(16), CGG(6) | CGT | −22 | 12.28 |
| GLU | GAG(19) | GAA | −19 | 10.64 |
| GLY | GGC(14), GGG(6) | GGT | −20 | 6.36 |
| LEU | CTC(15) | CTG | 0 | 11.4 |
| LYS | AAG(3) | AAA | −3 | 1.44 |
| PRO | CCC(8) | CCG | 0 | 6.16 |
| SER | AGC(11), TCG(5) | TCT | −16 | 5.02 |
| THR | ACG(5) | ACC | 0 | 2.4 |
| VAL | GTC(12), GTG(10) | GTT | −22 | 8.78 |

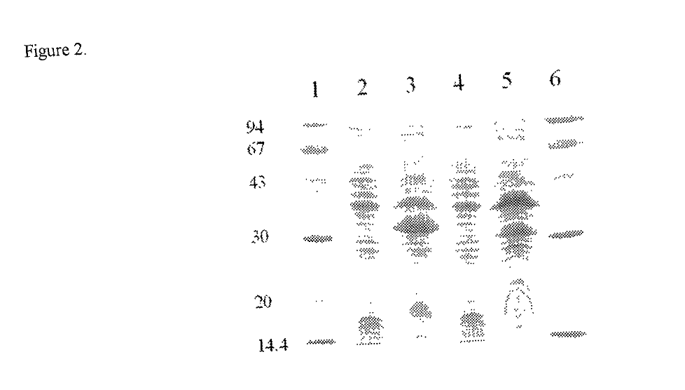

Induction of expression by IPTG in the pET-21(+) expression vector in the BL21 (IDE3) E. coli host resulted in the production of a ˜34 kDa polypeptide for 2,5-DKGR A and a ˜31 kDa polypeptide for 2,5-DKGR B (FIG. 2). The control cells with no added IPTG showed no such polypeptides. This level of expression indicates that 2,5-DKGR A and B represent the major proteins in the induced cells. The expression reached maximum levels within 4 hours after induction by IPTG. The purified polypeptides for 2,5-DKGR A and B exhibit enzyme activity towards both dihydroxy acetone phosphate and 2,5-DKG substrate.

Experimental Procedure

Pwo DNA polymerase and T4 DNA Ligase were obtained from Boehringer Mannheim Co. (Indianapolis, Ind.). Subcloning vector pFASTBACI, restriction enzymes (Nde I, Hind III, and Stu I), Calf Intestinal Alkaline Phosphatase (CIAP), and T4 Polynucleotide Kinase were obtained from New England Biolabs or GIBCO BRL (Gaitherburg, Md.). Expression vector pET-21 a(+) was from Novagen (Madison, Wis.). E.coli strains DH5a and BL21(DE3) were obtained from GIBCO BRL. Long oligonucleotides (˜60 nucleotides) were synthesized and further purified using polyacrylamide gel electrophoresis (PAGE) by Integrated DNA Technologies, Inc. Short oligonucleotides (˜20 oligonucleotides) were synthesized by the Bioanalysis Sequencing and Synthesis Laboratory at the Florida State University. QuikChange™ Site-Directed Mutagenesis Kit was purchased from Stratagene (La Jolla, Calif.).

Design of the Synthetic 2,5-DKGR A and B Nucleic Acid Sequences

Four general criteria were included in the design of synthetic sequences for 2,5-DKGR A and B, as follows. 1) Nucleotide sequences for 2,5-DKGR A and B were chosen to maintain the amino acid sequence as deduced from the wild-type nucleotide sequences (Anderson, Marks et al. 1985; Powers 1996). 2) In the case of amino acids with degenerate codons, codons were chosen to minimize G/C content at the wobble position. 3) Codons were chosen to maximize observed codon bias in enteric bacteria (Grosjean and Fiers 1982). However, in cases where the preferred (A/T-rich) codon(s) had poor bias in enteric bacteria (e.g. <0.1) preferred codons were chosen over A/T rich codons. 4) The cut-off limit of acceptable free energies for hairpin, dimerization and false priming for 60 mer test oligonucleotides were −7.0, −13, and −23 kcal/mol, respectively. Regions of possible hairpin formation, false priming and primer dimerization within the synthetic nucleotide sequences were identified and ranked by free energy calculations using the program Primer Premier (Premier Biosoft International). Based on the above criteria, a total of 20 oligonucleotides, each approximately 60 nucleotides long, were synthesized for the construction of both 2,5-DKGR A and B nucleic acid sequences. For construction purposes, these long oligonucleotides were designed with regions of complementary overlap (˜20 bases in length) with neighboring oligonucleotides.

Construction of Synthetic 2,5-DKGR A and B Nucleic Acid Sequences

A two-step PCR method was used for the construction of the synthetic 2,5-DKGR A and B nucleic acid sequences (Dillon and Rosen 1990). Template DNAs corresponding to the full-length synthetic nucleic acid sequences were generated using the complete set of 20 overlapping long oligonucleotides in a single PCR. Non-phosphorylated oligonucleotides (each 50 pmol), dNTPs (50 mM), Pwo polymerase (5 units) and PCR reaction buffer were mixed together in a 100 ml sample. The assembled nucleic acid sequences from this initial PCR were used as templates in a second PCR using phosphorylated outer primers. Templates (1 ul of first PCR reaction), dNTPs (20 mM), primers (each 20 pmol), Pwo polymerase (2.5 units) and PCR reaction buffer were mixed together in a 100 ml sample. Both PCR reactions were carried out in a Pelkin-Elmer thermal cycler for 30 cycles. Each cycle comprised denaturation, annealing and extension conditions of 94° C. for 1 minute, 60° C. for 1 minute, and 72° C. for 1 minute, respectively. An initial denaturation step of 94° C. for 5 min was applied for each PCR reaction. Polynucleotide products from both the first and second PCR steps were analyzed using ethidium bromide stained 1% Agarose gel electrophoresis.

Subcloning into Heterologous Expression Vector

Following the second PCR amplification, the 2,5-DKGR A and B nucleic acid sequences were extracted from agarose gel and subcloned into Stu I digested, and calf intestinal phosphatase treated, pFASTBAC1 vector (GIBCO BRL) via blunt end ligation. The choice of pFASTBAC1 for this step of subcloning was simply to expedite subsequent subcloning via restriction by Nde I and Hind III endonucleases. The synthetic nucleic acids for both 2,5-DKGR A and B were sequenced after subcloning into pFASTBAC1 by vector-specific primers. The synthetic nucleic acid sequences were restricted from the pFASTBAC1 vector using Nde I and Hind III restriction endonucleases and purified using 1% Agarose gel electrophoresis. The gel-extracted DNA fragments were ligated with Nde I/Hind III restricted pET-21a(+) expression vector (Novagen). After this final subcloning step, both genes were sequenced again in the pET-21a(+) vector to confirm their sequence.

Heterologous Expression in E. coli

2,5-DKGR A and B sequences in the pET-21a(+) expression vector were transformed into Escherichia coli strain BL21 (DE3). The transformed E. coli was grown at 37° C. in M9 minimal media (Sambrook, Fritsch et al. 1989) to an optical density of A⁶⁰⁰=1.2, at which point the temperature was shifted to 28° C. and expression of the synthetic 2,5-DKGR A and B sequences was induced by the addition of 1 mM isopropyl-b-D-thiogalactopyranoside (IPTG). The cells were allowed to grow for an additional 4.0 h and were then harvested by centrifugation (8,000× g for 10 min). The cell pastes were stored frozen at −20° C. before use. Induction of 2,5-DKGR A and B polypeptides was evaluated using sodium dodecylsulfate (SDS) PAGE.

Discussion

With the exception of AGC to TCT mutations for the codon corresponding to serine (5 total in 2,5-DKGR A and 11 in 2,5-DKGR B) all mutations in design of the synthetic nucleic acid sequences disclosed herein comprised point mutations at the codon wobble position. The greatest contribution to changes in GC content for 2,5-DKGR A included alanine, valine, glycine, glutamate, arginine, serine and lysine codons, as seen in Table 1.

A similar analysis for 2,5-DGKR B identifies valine, arginine, alanine, glycine, glutamate, and serine codons, shown in Table 2. Codon changes that did not affect GC content, but did improve codon bias for heterologous expression in E. coli, included leucine, proline and threonine codons for both 2,5-DKGR A and B, shown in Tables 1 and 2.

The 2-step PCR method used here to produce synthetic 2,5-DKGR A and B genes has been applied in the construction of a variety of genes, gene libraries, and plasmids (Rauscher, Morris et al. 1990; Ye, Johnson et al. 1992; Stemmer, Crameri et al. 1995). DNA sequences in the range of a ˜200 bp to 5 Kb can be assembled from chemically synthesized oligonucleotides in a single reaction (Stemmer, Crameri et al. 1995). However, the construction of synthetic 2,5-DKGR A and B nucleic acid sequences using the described two-step PCR method did not result in sequences free from sequence errors. In several different experiments we observed between one and five point mutations in the final PCR product. These mutations may be the result of long PCR reactions (Stemmer, Crameri et al. 1995). Barnes et al. has suggested that the addition of a proofreading polymerase may be important to ensure efficient long PCR reactions by combining high processivity with proofreading (Barnes 1994). However, it has been demonstrated that similar mutations were found with or without proofreading polymerase (Chen, Choi et al. 1994). The most expedient approach to obtain a correct sequence did not appear to be repeating the PCR steps, but to perform site-specific mutagenesis on the incorrect full-length synthetic sequences. Similar results have been noted by other groups using this method (Beattie and Fowler 1991).

Another approach previously used for the construction of synthetic genes involves annealing/ligation protocol of oligonucleotides comprising the entire sequence of a desired gene (Sproat and Gait 1985; Wosnick, Barnett et al. 1989; Climie and Santi 1990). In this method, oligonucleotides are annealed in a piecemeal fashion followed by joining with T4 DNA ligase.

By contrast, the approach employed herein has advantages over the annealing/ligation method. First, the two-step PCR method can be completed within 1 working day, however, annealing and ligation of overlapping sets of complementary oligonucleotides often require considerably longer time periods (i.e. weeks) to complete (Beattie and Fowler 1991). Another advantage of the present method is that it is more economical than annealing/ligation methods (Di Donato, de Nigris et al. 1993). A total of 20 oligonucleotides (˜60 mers) were used to construct both 2,5-DKGR A (834 bases) and B (831 bases) synthetic sequences. The number of bases involved is approximately 25% lower than the number of bases required by the established methodology of total synthesis using ligation of complementary oligonucleotides.

A particular goal in the development of synthetic nucleic acid sequences for 2,5-DKGR A and B was to improve the ability to perform polymerase-based methodologies, including PCR, mutagenesis and sequencing. Prior reports describing sequencing or mutagenesis efforts with 2,5-DKGR A or B have detailed problems with polymerase-based sequencing and PCR (Anderson, Marks et al. 1985; Powers 1996). In our own hands, the sequencing of wild-type 2,5-DKGR A has been very difficult to achieve—requiring proprietary commercial sequencing reagents and methods. Since the method utilized for the construction of the synthetic 2,5-DKGR nucleic acid sequences relies upon PCR under standard buffer conditions, the successful construction of a full-length sequence indicates that the problems associated with PCR and the wild-type genes have been substantially eliminated. Furthermore, the sequencing of the resulting synthetic 2,5-DKGR A and B sequences proceeds without the difficulty experienced with natural wild-type genes. The results indicate that the high GC content of 2,5-DKGR A and B contributes to problematic polymerase-based methodologies, and that appropriate reduction in GC content can solve this problem.

A further goal in the development of synthetic nucleic acid sequences for 2,5-DKGR A and B was to allow high-levels of expression in an E. coli host. SDS PAGE of the IPTG-induced BL21(IDE3) E. coli host indicates that high levels of expression of both 2,5-DKGR A and B are achieved (FIG. 2) with the synthetic sequences. Acetobacter species has been previously reported for the heterologous expression of 2,5-DKGR A primarily because expression in E. coli has proven unsuccessful (D. Powers, personal communication). Heterologous expression of 2,5-DKGR B in E. coli has been reported, but the levels of expression were not detailed (Grindley, Payton et al. 1988). In our hands, we also were never able to successfully employ the PCR method on the wild-type 2,5-DKGR A or B nucleic acid sequences for subcloning purposes, thus, we were unable to construct and evaluate expression of the wild-type gene sequence. The results disclosed here demonstrate that high-level heterologous expression of synthetic 2,5-DKGR A and B nucleic acid sequences has been achieved in E. coli, presumably due to the improvement in codon bias for enteric bacteria. Additional experiments with heterologous expression of the synthetic 2,5-DKGR A sequence indicate that approximately 30 mg of purified active protein can be isolated from 1.0 liter of bacterial culture in M9 minimal media. Although problematic in vitro polymerase-based procedures can sometimes be obviated by the inclusion of various additives in the reaction mixture (Baskaran, Kandpal et al. 1996), and improved heterologous expression can be achieved in hosts containing supplemental tRNA's for rare codons (Carstens and Waesche 1999), the development of the synthetic sequences of the present invention eliminates both of these restrictions. Due to the characteristically high GC content at the wobble position, the present methodology represents a generally applicable approach to allow efficient polymerase-based manipulation, as well as efficient heterologous expression of Corynebacterium nucleic acid sequences.

Preferred Embodiments of the Present Invention

Accordingly, the invention herein discloses an isolated nucleic acid comprising a degenerate variant of the nucleotide sequence of SEQ ID NO:1 (wild-type DKGR A gene) having a GC content from about 55% to about 67%. The GC content of the nucleic acid is effective for enhancing heterologous expression of the nucleic acid in enteric bacteria, and particularly in E. coli. Furthermore, the invention includes a nucleic acid sequence having wobble position GC content effective for enhancing the heterologous expression in Escherichia coli of a polypeptide encoded by the nucleic acid, that is, the polypeptide comprising the enzymes DKGR A and DKGR B. The nucleic acid disclosed further comprises a plurality of codons having a substitute base at a wobble position, wherein the plurality of codons is selected from the group of codons encoding alanine, arginine, glutamate, glycine, and valine. The substitute base is preferably effective for reducing overall GC content of the nucleic acid. In the nucleic acid of the invention wobble position GC content is effective for enhancing efficiency of the nucleic acid in a polymerase-based methodology, the methodology preferably including PCR, mutagenesis, and sequencing. Additionally, the synthetic nucleic acid further comprises an expression vector operably linked to an expression control sequence, wherein an isolated cell comprises the nucleic acid and the expression vector therefor operably linked to an expression control sequence. In the invention, the expression vector wherein the nucleic acid is operably linked to an expression control sequence, and an isolated cell or a progeny of the cell is transfected with the vector.

The invention set forth above may also be described as an isolated nucleic acid comprising a sequence having a GC content of from about 55% to about 67% and encoding a polypeptide having the amino acid sequence of SEQ ID NO:5, which represents wild-type DKGR A. The nucleic acid of the invention encoding DKGR A has a GC content effective for producing an average codon bias in enteric bacteria of from greater than about 44% up to about 66% so as to thereby enhance heterologous expression thereof, preferably in enteric bacteria, and most preferably in E. coli.

Another aspect of the invention includes an isolated nucleic acid comprising a degenerate variant of the nucleotide sequence of SEQ ID NO:3, which is the sequence for wild-type DKGR B, and having a GC content from about 56% to about 70%. As with the DKGR A sequence described above, this nucleic acid sequence has a GC content effective for enhancing heterologous expression of the nucleic acid in enteric bacteria. The DKGR B nucleotide sequence GC content being modified at wobble position bases to thereby enhance heterologous expression in Escherichia coli of a polypeptide encoded by the nucleic acid. This nucleic acid preferably comprises a plurality of codons having a substitute base at a wobble position, wherein the plurality of codons is selected from the group of codons encoding alanine, arginine, glutamate, glycine, and valine. In this method the substitute base is preferably effective for reducing GC content of the nucleic acid. The wobble position GC content is also effective for enhancing efficiency of a polymerase-based methodology with the nucleic acid, the methodology being selected from PCR, mutagenesis, and sequencing. The nucleic acid sequence for DKGR B, as shown in SEQ ID NO:3, may further comprise an expression vector operably linked to an expression control sequence, or an isolated cell comprising the nucleic acid and an expression vector therefor operably linked to an expression control sequence. The isolated cell may also comprise the nucleic acid according to SEQ ID NO:3 operably linked to an expression control sequence. As noted above, the synthetic nucleic acid of DKGR B may comprise an expression vector wherein the nucleic acid is operably linked to an expression control sequence, and wherein an isolated cell or a progeny of the cell is transfected with the vector.

The synthetic nucleic acid sequence for DKGR B has a GC content of from about 56% to about 70% and encodes a polypeptide having the amino acid sequence of SEQ ID NO:6, which is the wild-type enzyme. This nucleic acid has wobble position GC content effective for enhancing heterologous expression in Escherichia coli of the polypeptide encoded by the nucleic acid, which is wild-type DKGR B. The nucleic acid sequence additionally comprises a plurality of codons having a substitute base at a wobble position, the plurality of codons being selected from the group of codons encoding alanine, arginine, glutamate, glycine, and valine. As previously noted, the substitute base is preferably effective for reducing overall GC content of the nucleic acid. Wobble position GC content is effective for enhancing efficiency of a polymerase-based methodology with the nucleic acid, the polymerase-based methodology being selected from PCR, mutagenesis, and sequencing. This nucleic acid additionally may comprise an expression vector operably linked to an expression control sequence, and an isolated cell comprising the nucleic acid and an expression vector therefor operably linked to an expression control sequence, and an isolated cell comprises the nucleic acid operably linked to an expression control sequence. The synthetic nucleic acid sequence encoding wild-type DKGR B may further comprise an expression vector wherein the nucleic acid is operably linked to an expression control sequence, and wherein an isolated cell or a progeny of the cell is transfected with the vector. The GC content disclosed for the nucleic acid sequence encoding DKGR B is effective for producing an average codon bias in enteric bacteria of from greater than about 41% to about 68% so as to thereby enhance heterologous expression thereof.

Method aspects of the invention include a method of making a nucleic acid sequence encoding a polypeptide according to SEQ ID NO:5 (wild-type DKGR A enzyme) and having enhanced efficiency in a polymerase-based methodology, the method comprising synthesizing a degenerate variant of a nucleic acid sequence according to SEQ ID NO:1 (wild-type DKGR A gene) wherein a plurality of codons comprises at least one base substitution effective for sufficiently reducing GC content of the degenerate variant nucleic acid sequence to thereby enhance efficiency of the polymerase-based methodology. In the method, the polymerase-based methodology may be selected from PCR, mutagenesis, and sequencing.

Another method includes making a polypeptide, comprising culturing an isolated cell transfected with a synthetic nucleic acid comprising a degenerate variant of the nucleotide sequence of SEQ ID NO:1 (wild-type DKGR A gene) having a GC content of from about 55% to about 67% (such as for example synthetic DKGR A gene shown in SEQ ID NO:2), and an expression vector therefor operably linked to an expression control sequence, wherein culturing is effected under conditions permitting expression of the nucleic acid so as to produce a polypeptide encoded thereby. The polypeptide may be purified from the cell or from the medium.

A further method of making a polypeptide comprises culturing an isolated cell transfected with a synthetic nucleic acid comprising a sequence having a GC content of from about 55% to about 67% encoding a polypeptide having the amino acid sequence of SEQ ID NO:5 (wild-type DKGR A enzyme), and an expression vector therefor operably linked to an expression control sequence, wherein culturing comprises conditions permitting expression to produce the polypeptide. As noted above, the polypeptide is preferably purified from the cell or from the medium.

Yet another method of making a polypeptide includes culturing an isolated cell transfected with a synthetic nucleic acid comprising a sequence having a GC content of from about 55% to about 67% encoding a polypeptide having the amino acid sequence of SEQ ID NO:5, and an expression vector therefor operably linked to an expression control sequence, wherein culturing comprises conditions permitting expression to produce the polypeptide. In this method the polypeptide is also preferably purified from the cell or from the medium.

A polypeptide according to SEQ ID NO:5 (wild-type DKGR A enzyme) having enhanced expression in an enteric bacterium is made by the method comprising synthesizing a degenerate variant of a nucleic acid sequence encoding the polypeptide, wherein a plurality of codons comprises a base substitution preferably effective for reducing overall GC content in the nucleic acid sequence at a plurality of wobble position bases, and expressing the nucleic acid sequence in the enteric bacterium under conditions effective for production of the polypeptide encoded thereby. In the method, the enteric bacterium preferably comprises Escherichia coli.

A method of making vitamin C is also included in the invention, the method comprising the reduction of 2,5-diketo-D-gluconic acid to 2-keto-L-gulonic acid by a polypeptide according to SEQ ID NO:5 expressed from a nucleic acid comprising a degenerate variant of the nucleotide sequence of SEQ ID NO:1 (wild-type DKGR A gene) having a GC content of from about 55% to about 67%.

The methods herein above described are also equally practicable with a synthetic nucleotide sequence for DKGR B and with the polypeptide expressed therefrom. Accordingly, a method of making a nucleic acid sequence encoding a polypeptide according to SEQ ID NO:6 (wild-type DKGR B enzyme) and having enhanced efficiency in a polymerase-based methodology, comprises synthesizing a degenerate variant of a nucleic acid sequence according to SEQ ID NO:3 (wild-type DKGR B gene) wherein a plurality of codons comprises at least one base substitution effective for sufficiently reducing GC content of the degenerate variant nucleic acid sequence to thereby enhance efficiency of the polymerase-based methodology. In the method, the polymerase-based methodology is preferably selected from PCR, mutagenesis, and sequencing.

A method of making a polypeptide having the wild-type amino acid sequence of DKGR B according to SEQ ID NO:6 (wild-type DKGR B enzyme), comprises culturing an isolated cell transfected with a synthetic nucleic acid comprising a degenerate variant of the nucleotide sequence of SEQ ID NO:3 (wild-type DKGR B gene) having a GC content of from about 56% to about 70%, and an expression vector therefor operably linked to an expression control sequence, wherein culturing is effected under conditions permitting expression of the nucleic acid so as to produce a polypeptide encoded thereby. As in the other methods, the polypeptide produced may be purified from the cell or from the medium.

Yet an additional method of making a polypeptide includes culturing an isolated cell transfected with a synthetic nucleic acid comprising a sequence having a GC content of from about 56% to about 70% encoding a polypeptide having the amino acid sequence of SEQ ID NO:6 (wild-type DKGR B enzyme), and an expression vector therefor operably linked to an expression control sequence, wherein culturing comprises conditions permitting expression to produce the polypeptide. Similarly to the methods set forth above, the polypeptide may preferably be purified from the cell or from the medium.

Yet a further method of making a polypeptide according to SEQ ID NO:6 (wild-type DKGR B enzyme) having enhanced expression in an enteric bacterium comprises synthesizing a degenerate variant of a nucleic acid sequence encoding the polypeptide, wherein a plurality of codons comprises a base substitution preferably effective for reducing the overall GC content in the nucleic acid sequence in a plurality of wobble position bases; and expressing the nucleic acid sequence in the enteric bacterium under conditions effective for production of the polypeptide encoded thereby. As noted above, in this method, a preferred enteric bacterium is Escherichia coli.

Also, a method of making vitamin C comprises the reduction of 2,5-diketo-D-gluconic acid to 2-keto-L-gulonic acid by a polypeptide having a sequence according to SEQ ID NO:6 (wild-type DKGR B enzyme) expressed from a nucleic acid comprising a degenerate variant of the nucleotide sequence of SEQ ID NO:3 (wild-type DKGR B gene) having a GC content of from about 56% to about 70%.

Finally, a method of making a nucleic acid sequence encoding a polypeptide having a wild type amino acid sequence according to SEQ ID NO:1 (wild-type DKGR A gene) or SEQ ID NO:3 and enhanced heterologous expression in enteric bacteria, comprises synthesizing a degenerate variant of the nucleic acid sequence wherein a plurality of codons comprises a base substitution effective for reducing GC content at a wobble position. In this method, the GC reduction is preferably made in a plurality of codon wobble positions, and Escherichia coli is the preferred enteric bacteria.

In the drawings and specification, there have been disclosed a typical preferred embodiment of the invention, and although specific terms are employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

That which is claimed:

1. An isolated nucleic acid comprising a degenerate variant of the nucleotide sequence of SEQ ID NO:1 having a GC content from about 55% to about 67%.

2. The nucleic acid of claim 1, wherein GC content is effective for enhancing heterologous expression of said nucleic acid in enteric bacteria.

3. The nucleic acid of claim 1, further comprising a plurality of codons having a substitute base at a wobble position, said plurality of codons selected from the group of codons encoding alanine, arginine, glutamate, glycine, and valine.

4. The nucleic acid of claim 1, wherein wobble position GC content is effective for enhancing efficiency of a polymerase-based methodology with said nucleic acid.

5. The nucleic acid of claim 4, wherein said polymerase-based methodology is selected from PCR, mutagenesis, and sequencing.

6. The nucleic acid of claim 1, further comprising an expression vector operably linked to an expression control sequence.

7. The nucleic acid of claim 1, wherein an isolated cell comprises said nucleic acid and an expression vector therefor operably linked to an expression control sequence.

8. The nucleic acid of claim 1, wherein an isolated cell comprises said nucleic acid operably linked to an expression control sequence.

9. The nucleic acid of claim 1, further comprising an expression vector wherein said nucleic acid is operably linked to an expression control sequence, and wherein an isolated cell or a progeny of said cell is transfected with said vector.

10. An isolated nucleic acid comprising a sequence having a GC content of from about 55% to about 67% and encoding a polypeptide having the amino acid sequence of SEQ ID NO:5.

11. The nucleic acid of claim 10, further comprising a plurality of codons having a substitute base at a wobble position, said plurality of codons selected from the group of codons encoding alanine, arginine, glutamate, glycine, and valine.

12. The nucleic acid of claim 10, wherein wobble position GC content is effective for enhancing efficiency of a polymerase-based methodology with said nucleic acid.

13. The nucleic acid of claim 12, wherein said polymerase-based methodology is selected from PCR, mutagenesis, and sequencing.

14. The nucleic acid of claim 10, further comprising an expression vector operably linked to an expression control sequence.

15. The nucleic acid of claim 10, wherein an isolated cell comprises said nucleic acid and an expression vector therefor operably linked to an expression control sequence.

16. The nucleic acid of claim 10, wherein an isolated cell comprises said nucleic acid operably linked to an expression control sequence.

17. The nucleic acid of claim 10, further comprising an expression vector wherein said nucleic acid is operably linked to an expression control sequence, and wherein an isolated cell or a progeny of said cell is transfected with said vector.

18. The nucleic acid of claim 10, wherein said GC content is effective for producing an average codon bias in enteric bacteria of from greater than about 44% up to about 66% so as to thereby enhance heterologous expression thereof.

19. An isolated nucleic acid comprising a degenerate variant of the nucleotide sequence of SEQ ID NO:3 having a GC content from about 56% to about 70%.

20. The nucleic acid of claim 19, wherein GC content is effective for enhancing heterologous expression of said nucleic acid in enteric bacteria.

21. The nucleic acid of claim 19, further comprising a plurality of codons having a substitute base at a wobble position, said plurality of codons selected from the group of codons encoding alanine, arginine, glutamate, glycine, and valine, said substitute base effective for reducing GC content of said nucleic acid.

22. The nucleic acid of claim 19, wherein wobble position GC content is effective for enhancing efficiency of a polymerase-based methodology with said nucleic acid.

23. The nucleic acid of claim 22, wherein said polymerase-based methodology is selected from PCR, mutagenesis, and sequencing.

24. The nucleic acid of claim 19, further comprising an expression vector operably linked to an expression control sequence.

25. The nucleic acid of claim 19, wherein an isolated cell comprises said nucleic acid and an expression vector therefor operably linked to an expression control sequence.

26. The nucleic acid of claim 19, wherein an isolated cell comprises said nucleic acid operably linked to an expression control sequence.

27. The nucleic acid of claim 19, further comprising an expression vector wherein said nucleic acid is operably linked to an expression control sequence, and wherein an isolated cell or a progeny of said cell is transfected with said vector.

28. An isolated nucleic acid comprising a sequence having a GC content of from about 56% to about 70% and encoding a polypeptide having the amino acid sequence of SEQ ID NO:6.

29. The nucleic acid of claim 28, further comprising a plurality of codons having a substitute base at a wobble position, wherein said substitute base is effective for enhancing heterologous expression in Escherichia coli of a polypeptide encoded by said nucleic acid.

30. The nucleic acid of claim 29, wherein said plurality of codons is selected from the group of codons encoding alanine, arginine, glutamate, glycine, and valine.

31. The nucleic acid of claim 28, wherein wobble position GC content is effective for enhancing efficiency of a polymerase-based methodology with said nucleic acid.

32. The nucleic acid of claim 31, wherein said polymerase-based methodology is selected from PCR, mutagenesis, and sequencing.

33. The nucleic acid of claim 28, further comprising an expression vector operably linked to an expression control sequence.

34. The nucleic acid of claim 28, wherein an isolated cell comprises said nucleic acid and an expression vector therefor operably linked to an expression control sequence.

35. The nucleic acid of claim 28, wherein an isolated cell comprises said nucleic acid operably linked to an expression control sequence.

36. The nucleic acid of claim 28, further comprising an expression vector wherein said nucleic acid is operably linked to an expression control sequence, and wherein an isolated cell or a progeny of said cell is transfected with said vector.

37. The nucleic acid of claim 28, wherein said GC content is effective for producing an average codon bias in enteric bacteria of from greater than about 41% to about 68% so as to thereby enhance heterologous expression thereof.

38. A method of making a nucleic acid sequence encoding a polypeptide according to SEQ ID NO:5 and having enhanced efficiency in a polymerase-based methodology, the method comprising synthesizing a degenerate variant of a nucleic acid sequence according to SEQ ID NO:1 wherein a plurality of codons comprises at least one base substitution effective for sufficiently reducing GC content of said degenerate variant nucleic acid sequence to thereby enhance efficiency of the polymerase-based methodology.

39. The method of claim 38, wherein the polymerase-based methodology is selected from PCR, mutagenesis, and sequencing.

40. A method of making a polypeptide, comprising culturing an isolated cell transfected with a synthetic nucleic acid comprising a degenerate variant of the nucleotide sequence of SEQ ID NO:1 having a GC content of from about 55% to about 67%, and an expression vector therefor operably linked to an expression control sequence, wherein culturing is effected under conditions permitting expression of said nucleic acid so as to produce a polypeptide encoded thereby.

41. The method of claim 40, further comprising purifying the polypeptide from the cell or from the medium.

42. A method of making a polypeptide, the method comprising culturing an isolated cell transfected with a synthetic nucleic acid comprising a sequence having a GC content of from about 55% to about 67% encoding a polypeptide having the amino acid sequence of SEQ ID NO:5, and an expression vector therefor operably linked to an expression control sequence, wherein culturing comprises conditions permitting expression to produce the polypeptide.

43. The method of claim 42, further comprising purifying the polypeptide from the cell or from the medium.

44. A method of making a polypeptide according to SEQ ID NO:5 having enhanced expression in an enteric bacterium, the method comprising:
synthesizing a degenerate variant of a nucleic acid sequence encoding the polypeptide, wherein a plurality of codons comprises a base substitution at a wobble position effective for reducing GC content in the nucleic acid sequence; and
expressing the nucleic acid sequence in the enteric bacterium under conditions effective for production of the polypeptide encoded thereby.

45. The method of claim 44, wherein the enteric bacterium comprises Escherichia coli.

46. A method of making vitamin C, comprising the reduction of 2,5-diketo-D-gluconic acid to 2-keto-L-gulonic acid by a polypeptide according to SEQ ID NO:5 expressed from a nucleic acid comprising a degenerate variant of the nucleotide sequence of SEQ ID NO:1 having a GC content of from about 55% to about 67%.

47. A method of making a nucleic acid sequence encoding a polypeptide according to SEQ ID NO:6 and having enhanced efficiency in a polymerase-based methodology, the method comprising synthesizing a degenerate variant of a nucleic acid sequence according to SEQ ID NO:3 wherein a plurality of codons comprises at least one base substitution effective for sufficiently reducing GC content of said degenerate variant nucleic acid sequence to thereby enhance efficiency of the polymerase-based methodology.

48. The method of claim 47, wherein the polymerase-based methodology is selected from PCR, mutagenesis, and sequencing.

49. A method of making a polypeptide, comprising culturing an isolated cell transfected with a synthetic nucleic acid comprising a degenerate variant of the nucleotide sequence of SEQ ID NO:3 having a GC content of from about 56% to about 70%, and an expression vector therefor operably linked to an expression control sequence, wherein culturing is effected under conditions permitting expression of said nucleic acid so as to produce a polypeptide encoded thereby.

50. The method of claim 49, further comprising purifying the polypeptide from the cell or from the medium.

51. A method of making a polypeptide, the method comprising culturing an isolated cell transfected with a synthetic nucleic acid comprising a sequence having a GC content of from about 56% to about 70% encoding a polypeptide having the amino acid sequence of SEQ ID NO:6, and an expression vector therefor operably linked to an expression control sequence, wherein culturing comprises conditions permitting expression to produce the polypeptide.

52. The method of claim 51, further comprising purifying the polypeptide from the cell or from the medium.

53. A method of making a polypeptide according to SEQ ID NO:6 having enhanced expression in an enteric bacterium, the method comprising:
synthesizing a degenerate variant of a nucleic acid sequence encoding the polypeptide, wherein a plurality of codons comprises a base substitution at a wobble position effective for reducing GC content in the nucleic acid sequence; and
expressing the nucleic acid sequence in the enteric bacterium under conditions effective for production of the polypeptide encoded thereby.

54. The method of claim 53, wherein the enteric bacterium comprises Escherichia coli.

55. A method of making vitamin C, comprising the reduction of 2,5-diketo-D-gluconic acid to 2-keto-L-gulonic acid by a polypeptide expressed from a nucleic acid comprising a degenerate variant of the nucleotide sequence of SEQ ID NO:3 having a GC content of from about 56% to about 70%.

56. A method of making a nucleic acid sequence encoding a polypeptide having a wild type amino acid sequence according to SEQ ID NO:1 or SEQ ID NO:3 and enhanced heterologous expression in enteric bacteria, the method comprising synthesizing a degenerate variant of the nucleic acid sequence wherein a plurality of codons comprises a base substitution effective for reducing GC content.

57. The method of claim 56, wherein the GC reduction is made in a plurality of codon wobble positions.

58. The method of claim 56, wherein Escherichia coil is the enteric bacteria.
